(19) United States
(12) Patent Application Publication
(10) Pub. No.: US 2011/0230666 A1
(43) Pub. Date: Sep. 22, 2011

(54) PROCESS FOR THE SEPARATION OF ENANTIOMERICALLY PURE COMPOUNDS

(75) Inventors: Thota Giridhar, Hyderabad (IN); Gudipati Srinivasulu, Hyderabad (IN); Kotaru Srinivasa Rao, Hyderabad (IN)

(73) Assignee: SHODHANA LABORATORIES LIMITED, HYDERABAD (IN)

(21) Appl. No.: 13/131,518

(22) PCT Filed: Feb. 5, 2009
(86) PCT No.: PCT/IN09/00092

§ 371 Date: May 26, 2011

(30) Foreign Application Priority Data
Jun. 3, 2008 (IN) 1358/CHE/2008

(51) Int. Cl. C07D 333/36; C07C 269/08; C07C 209/88; C07D 307/87

(52) U.S. Cl. 549/75; 560/159; 564/302; 549/467

(57) ABSTRACT

The present patent application relates to an improved process for the separation of enantiomerically pure compounds. Specifically it relates to separation of enantiomerically enriched Rivastigmine, Duloxetine, Escitalopram and their intermediates in high yields.

### FIELD OF THE INVENTION

The present patent application relates to an improved process for the separation of enantiomerically pure compounds. Specifically it relates to separation of enantiomerically pure Rivastigmine, Duloxetine, Escitalopram and their intermediates in high yields.

### BACKGROUND OF THE INVENTION

Some drug molecules are chiral and the enantiomers have different effects on biological entities. They can be sold as one enantiomer or as a racemic mixture. Examples include Thalidomide, Ibuprofen, and Salbutamol. In cases like Salbutamol and Thalidomide the inactive isomer may be harmful. Therefore, there is a need to obtain the required enantiomer of the drug molecule which is free of its enantiomeric impurity, and also free of other process related impurities

Rivastigmine hydrogentartrate is chemically known as (S)—N-Ethyl-N-methyl-3-[1-(dimethylamino)ethyl]-phenyl carbamate hydrogen-(2R,3R)-tartrate (hereinafter referred to as “Rivastigmine hydrogentartrate”) and has structural Formula I.

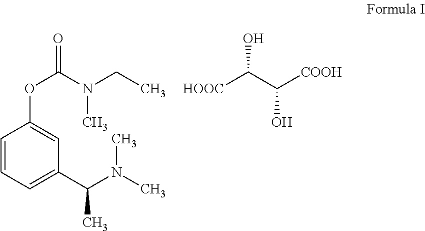

U.S. Pat. No. 4,948,807 describes the compound (S)—N-ethyl, N-methyl-3-[1-(dimethylamino)ethyl]phenyl carbamate and its pharmacologically acceptable salts along with a pharmaceutical composition useful for treating anti-cholinesterase activity in humans.

U.S. Pat. No. 5,602,176 describes (S)—N-ethyl-N-methyl-3-[(1-dimethylamino)ethyl]-phenyl carbamate in free base or acid addition salt form as useful for its anti-cholinesterase activity. It also describes process for preparation involving resolution of N-ethyl, N-methyl-3-[1-(dimethylamino)ethyl]phenyl carbamate in presence of (+)-di-para-toluoyl tartaric acid ((+)-DPTTA). The overall yield of the resolution process is very low and making the process not suitable for commercial manufacturing.

International Application Publication No. WO 04/037771 discloses preparation of Rivastigmine by resolving the intermediates to obtain an optically pure 3-[(1-dimethylamino)ethyl]-phenol and converting the same to Rivastigmine.

Duloxetine hydrochloride has the chemical name (S)-(+)-N-methyl-γ-(1-naphthyloxy)-2-thiophenepropylamine hydrochloride and is structurally represented by Formula II.

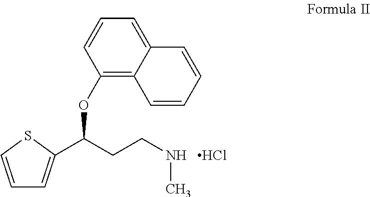

U.S. Pat. No. 5,023,269 describes N-methyl-3-(1-naphthalenyloxy)-3-(2-thienyl)propanamine oxalate, its related compounds and processes for their preparation.

Processes for preparation of duloxetine, its pharmaceutically acceptable salts and its intermediates have been described in: U.S. Pat. No. 5,362,886; European Patent No. 457559; International Application Publication Nos. WO 2006/071868. WO 2006/099468, and WO 2004/056795; U.S. Patent Application Publication Nos. 2006/0128791 and 2004/0249170; and Drugs of the Future 2000, 25(9) 907-916.

Escitalopram is chemically known as (S)-1-[3-(dimethylamino)propyl]-1-(4-fluorophenyl)-1,3-dihydro-5-isobenzofuran carbonitrile and described by the following structural Formula III.

U.S. Pat. No. 4,943,590 discloses Escitalopram, non-toxic acid addition salts thereof and processes for their preparation.

Processes for preparation of Escitalopram, its pharmaceutically acceptable salts and its intermediates have been described in: U.S. Pat. No. 6,762,307; International Application Publication Nos. WO 2006/106531, WO 2006/136169, and WO 2006/025071; U.S. Patent Application Publication Nos. 2006/0009515.

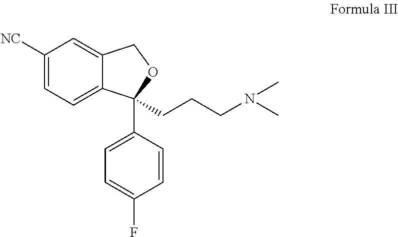

Resolution by diastereomeric salt formation is considered to be simplest process for the separation of enantiomerically pure compounds from their corresponding racemic mixtures. However, this technique suffers from a disadvantage that the yield of the required isomer obtained is less in most of the cases, apart from discarding the unwanted isomer.

Often this low yield of required isomer makes the process expensive and one needs to search for various procedures to improve the overall yield of the resolution process. Some times racemization of the unwanted isomer to its racemic mixture may not be feasible for some molecules due to its chemical structure and stability.

In general, the resolution technique includes reaction of racemic mixture with an optically pure acid or a base to form the diastereomeric salt as solid and recovering the required isomer from the diastereomeric salt. The mother liquors are generally discarded. But some times to improve the overall yield of the resolution, the mixture of isomer obtained from the mother liquors may be reacted with the same optically pure acid or base again, to form the diastereomeric salt and recover the required isomer as second crop as described in Flow Chart 3. The yield improvement obtained by the aforesaid process is also not significant, rendering the process not suitable for commercial manufacturing.

Therefore there is a need for a process, which is advantageous to increase the yields of the final product and also to yield an enantiomerically enriched form of drug molecules.

It is the surprising finding by the inventors of the present application that the process for the present application provides significant improvement in yield, which makes the process commercially viable.

### SUMMARY OF THE INVENTION

In one aspect, the present application provides a process for separation of the required isomer from a first mixture of isomers, which process includes:

a) reacting the first mixture of isomers with a first optically pure acid or base to recover the first diastereomeric salt of unwanted isomer as solid;

b) reacting the second mixture of isomers obtained from the mother liquors in step a) with a second optically pure acid or base having opposite rotation with respect to the first optically pure acid or base to form a second diastereomeric salt as solid; and

c) converting the second diastereomeric salt to the required isomer.

In a first embodiment of the present invention, there is provided a process for separation of Rivastigmine, from a first mixture of isomers which process includes:

a) reacting a first mixture of isomers with (−) DPTTA to recover (R)-Rivastigmine (−) DPTTA salt as solid;

b) reacting the second mixture of isomers obtained from the mother liquors in step a) with (+) DPTTA to form Rivastigmine (+) DPTTA salt as solid; and

c) converting the Rivastigmine (+) DPTTA salt to Rivastigmine or a pharmaceutically acceptable salt thereof.

In second embodiment, the present invention provides a process for separation of S-(−)-1-(3-methoxyphenyl)ethanamine from a first mixture of isomers, which process includes:

a) reacting the first mixture of isomers with D (−) Mandelic acid (MA) to recover R-(+)-1-(3-methoxyphenyl)ethanamine D (−) MA salt as solid;

b) reacting the second mixture of isomers obtained from the mother liquors in step a) with L (+) MA to form S-(−)-1-(3-methoxyphenyl)ethanamine L (+) MA salt as solid; and

c) converting the S-(−)-1-(3-methoxyphenyl)ethanamine L (+) MA salt to S-(−)-1-(3-methoxyphenyl)ethanamine.

In third embodiment, the present invention provides a process for separation of S-(−)-N,N-dimethyl-3-hydroxy-3-(2-thienyl)propanamine from a first mixture of isomers, which process includes:

a) reacting the first mixture of isomers with D (−) MA to recover R-(+)-N,N-dimethyl-3-hydroxy-3-(2-thienyl)propanamine D-(−) MA salt as solid;

b) reacting the second mixture of isomers obtained from the mother liquors of step a) with L (+) MA to form S-(−)-N,N-dimethyl-3-hydroxy-3-(2-thienyl)propanamine L (+) MA salt as solid; and

c) converting the S-(−)-N,N-dimethyl-3-hydroxy-3-(2-thienyl)propanamine L (+) MA salt to S-(−)-N,N-dimethyl-3-hydroxy-3-(2-thienyl)propanamine.

In fourth embodiment, the present application provides a process for separation of (−)4-(4-(dimethylamino)-1-(4-fluorophenyl)-1-hydroxybutyl)-3-(hydroxymethyl)benzonitrile (escitalopram diol) from a first mixture of isomers, which process includes

a) reacting the first mixture of isomers with (−) DPTTA to obtain (+)4-(4-(dimethylamino)-1-(4-fluorophenyl)-1-hydroxybutyl)-3-(hydroxymethyl)benzonitrile (−) DPTTA salt as solid;

b) reacting the second mixture of isomers obtained from the mother liquors of step a) with (+) DPTTA to form escitalopram diol (+) DPTTA salt as solid; and

c) converting the escitalopram diol (+) DPTTA salt to Escitalopram of Formula III or a salt thereof.

In another aspect the present invention relates to the method of using the enantiomerically pure intermediates obtained according to the process of present application in the preparation of active pharmaceutical ingredients including Rivastigmine, Duloxetine and Escitalopram or a salt thereof.

### DETAILED DESCRIPTION OF THE INVENTION

As used herein, the term “required isomer” denotes the enantiomerically pure isomer which is useful in the preparation of active pharmaceutical ingredients and pharmaceutical preparations; the term “unwanted isomer” refers to the enantiomerically pure isomer which is not useful in the preparation of active pharmaceutical ingredients and pharmaceutical preparations.

As used herein, the term “mixture of isomers” denotes the mixture of required and unwanted isomer of the specific compound in any ratio including the Racemic mixture, wherein the isomers are in equal ratios.

The terms “enantiomerically pure” or “optically pure” as used herein refers the chiral purity more than 95%, preferably more than 99% w/w.

As set forth above, in one aspect, the preset application provides a process for separation of the required isomer from a first mixture of isomers, which process includes:

a) reacting the first mixture of isomers with a first optically pure acid or base to recover the first diastereomeric salt of unwanted isomer as solid;

b) reacting the second mixture of isomers obtained from the mother liquors in step a) with a second optically pure acid or base having opposite rotation with respect to the first optically pure acid or base to form a second diastereomeric salt as solid; and

c) converting the second diastereomeric salt to the required isomer.

The first mixture of isomers that is used in the process of the present application is having about 50% or more by weight, preferably about 60% or more by weight, more preferably 70% or more by weight of the unwanted isomer.

The first mixture of isomers is obtained by a regular synthetic process or a stereo selective reaction or by processing the mother liquors obtained during the resolution of a racemic mixture using an optically pure acid or base and recovering the required isomer as diastereomeric salt. The mother liquors obtained during the recrystallization of the required diastereomeric salt may also be combined with the mother liquors obtained during the resolution of a racemic mixture.

Suitably the mother liquors may be processed directly or it may be concentrated to remove the existing solvent followed by addition of another suitable solvent.

Suitable solvent that can be used include water-immiscible solvents like halogenated solvents such as dichloromethane, dichloroethane, and chloroform; hydrocarbon solvents such as n-hexane, n-heptane, toluene, xylene and the like; ester solvents such as ethyl acetate, butyl acetate; ether solvents such diisopropyl ether, dibutyl ether, alcohol solvents such as methanol, ethanol, isopropanol, n-butanol and isobutanol, ketone solvents such as methyl ethyl ketone, methyl isobutyl ketone and mixtures thereof.

The mother liquors may be treated with an acid or base with or without water to obtain the free base or free acid of the respective compounds in the solution. The solution containing free base or free acid may be used directly in the reaction or it may be concentrated to remove the solvent.

Step a) involves reacting the first mixture of isomers with a first optically pure acid or base to recover the first diastereomeric salt of unwanted isomer as solid.

If the substrate (first mixture of isomers) is a base, then optically pure acid is used in the reaction and if the substrate is an acid, then optically pure base is used in the reaction.

The optically pure acid or base that is used in the process of step a) is selected depending on its ability to form diastereomeric salt of unwanted isomer as solid. Preferably, if the first mixture of isomers is obtained by resolution with an optically pure acid or base, the same optically pure acid or base, but having an opposite optical rotation may be selected.

Suitable optically pure acids include mandelic acid, tartaric acid, di-p-toluyl tartaric acid, dibenzoyl tartaric acid, camphor sulfonic acid and the like. Suitable optically pure bases include 1-phenylethylamine, ephedrine, 2-amino-1-butanol, 2-amino-1-phenyl-1,3-propanediol, 1-naphthyl-1-ethylamine, (−)-quinine, (+)-quinidine, (−)-brucine and (+)-dehydroabietylamine. Other suitable optically pure acids and bases may be determined by testing and the use thereof in a process as described above is also within the scope of the present application.

Suitably, the solvent employed is a lower alkanol, such as methanol, ethanol or isopropanol; ketone solvents such as acetone, methyl ethyl ketone or methyl isobutyl ketone. Although again other suitable solvents can be determined by testing and the use thereof in a process as described above falls within the scope of the present invention. A preferred solvent is methanol.

Suitable temperatures for conducting the reaction range from about 20° C. to 80° C., or preferably 25° C. to 35° C. The reaction can be conducted for about 30 minutes to about 5 hours, or the reaction conditions can be maintained as long as required for the complete reaction to form the desired product.

The solid product obtained is recovered from the reaction mixture by suitable techniques such as decantation, filtration by gravity or by suction, centrifugation, and the like. The crystals so isolated can be washed on with a solvent to wash out the mother liquor.

The wet cake thus obtained is discarded and the mother liquors containing the second mixture of isomers in the form of a salt with the optically pure acid or base can be converted to the free base or free acid by treating with a base or an acid respectively.

Step b) involves reacting the second mixture of isomers obtained from the mother liquors of step a) with a second optically pure acid or base having opposite rotation with respect to the first optically pure acid or base to form a second diastereomeric salt of required isomer as solid; and

The solvents, reagents and reaction conditions described in step a) are useful for step b) also except that the optically pure acid has the opposite rotation.

The solid product thus obtained is recovered from the reaction mixture by suitable techniques such as decantation, filtration by gravity or by suction, centrifugation, and the like. The crystals so isolated can carry a small proportion of occluded mother liquor containing a higher percentage of unwanted isomer. If desired, the crystals can be washed on with a solvent to wash out the mother liquor.

The wet cake obtained can be optionally further dried. Drying can be suitably carried out in a tray dryer, vacuum oven, air oven, fluidized bed drier, spin flash dryer, flash dryer and the like. The drying can be carried out at temperatures of about 35° C. to about 70° C. or even above where product permits for any desired time period to achieve a desired result, time from about 1 to 20 hours, or longer.

Step c) involves converting the second diastereomeric salt to the required isomer by treating with a base or an acid.

Suitable bases that can be used include but are not limited to: alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof.

Suitable acids that can be used include hydrochloric acid, sulfuric acid, acetic acid and the like.

These bases or bases can be used in their pure form or in the form of corresponding aqueous solutions

Suitably, aqueous solutions containing about 5% to 50%, or about 10% to 20%, (w/v) of the corresponding base or acid can be used.

Suitable solvents which can be used for extracting the required isomer from the aqueous mixture include, but are not limited to: esters such as ethyl acetate, n-propyl acetate, n-butyl acetate, t-butyl acetate and the like; hydrocarbons such as toluene, xylene, n-hexane, n-heptane, cyclohexane and the like; ether solvents such as diethyl ether, diisopropyl ether, methyl tertiary-butyl ether and the like; and mixtures thereof.

After reaction completion, the organic layer containing the required isomer is separated and may be progressed to further processing directly, or it can be concentrated to isolate the free acid or free base.

Suitably the free acid or free base of the required isomer obtained above can be converted to an active pharmaceutical ingredient or it's pharmaceutically acceptable salts by processes including those that are known in the art.

The process of the present application is explained in more detail, wherein optically pure acid is selected for resolution, in the following Flow Chart 1.

In a first embodiment of the present invention, there is provided a process for separation of Rivastigmine, from a first mixture of isomers which process includes:

a) reacting the first mixture of isomers with (−) DPTTA to recover the diastereomeric salt of (R)-Rivastigmine as solid;

b) reacting the second mixture of isomers obtained from the mother liquors in step a) with (+) DPTTA to form Rivastigmine (+) DPTTA salt as solid; and

c) converting the Rivastigmine (+) DPTTA salt to Rivastigmine.

The first mixture of isomers that is used in the process of the present application is having about 50% or more by weight, preferably about 60% or more by weight, more preferably 70% or more by weight of R-Rivastigmine.

The first mixture of isomers having about 60% or more by weight of R-Rivastigmine is obtained by processing the mother liquors obtained during the resolution of a racemic mixture with (+) DPTTA after recovering the Rivastigmine as diastereomeric salt. The mother liquors obtained during the recrystallization of the Rivastigmine diastereomeric salt are also combined with the mother liquors obtained during the resolution of a racemic mixture.

Suitably the mother liquors are concentrated to remove the existing solvent followed by addition of another suitable solvent.

Suitable solvent that can be used include water-immiscible solvents like halogenated solvents such as dichloromethane, dichloroethane, and chloroform; hydrocarbon solvents such as n-hexane, n-heptane, toluene, xylene and the like; ester solvents such as ethyl acetate, butyl acetate; ether solvents such diisopropyl ether, dibutyl ether, alcohol solvents such as methanol, ethanol, isopropanol, n-butanol and isobutanol, ketone solvents such as methyl ethyl ketone, methyl isobutyl ketone and mixtures thereof.

The mother liquors may be treated with a base with or without water to obtain the free base of the respective compounds in the solution. The solution containing free base may be used directly in the reaction or it may be concentrated to remove the solvent.

Step a) involves reacting the first mixture of isomers with (−) DPTTA to recover the diastereomeric salt of R-Rivastigmine as solid;

Suitably, the solvent employed is a lower alkanol, such as methanol, ethanol or isopropanol; ketone solvents such as acetone, methyl ethyl ketone or methyl isobutyl ketone. A preferred solvent is methanol.

The solid product obtained is recovered from the reaction mixture by suitable techniques such as decantation, filtration by gravity or by suction, centrifugation, and the like. The crystals so isolated can be washed on with a solvent to wash out the mother liquor.

The wet cake thus obtained is discarded and the mother liquors contain the second mixture of isomers in the form DPTTA salts can be converted to the free base by treating with a base.

Step b) involves reacting the second mixture of isomers obtained from the mother liquors of step a) with (+) DPTTA to form Rivastigmine (+) DPTTA salt as solid.

The solvents, reagents and reaction conditions descried in step a) are applicable for step b) also, except usage of (+) DPTTA in place of (−) DPTTA.

The solid product thus obtained is recovered from the reaction mixture by suitable techniques such as decantation, filtration by gravity or by suction, centrifugation, and the like. The crystals so isolated can carry a small proportion of occluded mother liquor containing a higher percentage of unwanted isomer. If desired, the crystals can be washed on with a solvent to wash out the mother liquor.

The wet cake obtained can be optionally further dried. Drying can be suitably carried out in a tray dryer, vacuum oven, air oven, fluidized bed drier, spin flash dryer, flash dryer and the like. The drying can be carried out at temperatures of about 35° C. to about 70° C. or above where product permits for any desired time period to achieve a desired result, times from about 1 to 20 hours, or longer.

Step c) involves converting the Rivastigmine (+) DPTTA salt to Rivastigmine.

Suitable bases that can be used include but are not limited to: alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof.

These bases can be used in their pure form or in the form of corresponding aqueous solutions

Suitably, aqueous solutions containing about 5% to 50%, or about 10% to 20%, (w/v) of the corresponding base can be used.

Suitable solvents which can be used for extracting the required isomer from the aqueous mixture include, but are not limited to: esters such as ethyl acetate, n-propyl acetate, n-butyl acetate, t-butyl acetate and the like; hydrocarbons such as toluene, xylene, n-hexane, n-heptane, cyclohexane and the like; ether solvents such as diethyl ether, diisopropyl ether, methyl tertiary-butyl ether and the like; and mixtures thereof.

After reaction completion, the organic layer containing the required isomer is separated and may be progressed to further processing directly, or it can be concentrated to isolate the free base.

Suitably the Rivastigmine obtained above can be converted into its pharmaceutically acceptable salts by processes including those that are known in the art.

The process of the present embodiment is explained in more detail, in the following Flow Chart 2.

The process of recovering the required isomer from a racemic mixture that is used conventionally prior to the process of the present application is explained in the following Flow Chart 3.

The present process results in overall improvement in the yield of the Rivastigmine. The yield improvement obtained by the process of the present application is evident from the information provided in the representative examples.

In second embodiment, the present invention provides a process for separation of S-(−)-1-(3-methoxyphenyl)ethanamine from a first mixture of isomers, which process includes:

a) reacting the first mixture of isomers with D-(−) Mandelic acid (MA) to recover the diastereomeric salt of R-(+)-1-(3-methoxyphenyl)ethanamine D-(−) MA as solid;

b) reacting the second mixture of isomers obtained from the mother liquors in step a) with L (+) MA to form S-(−)-1-(3-methoxyphenyl)ethanamine L (+) MA as solid; and

c) converting the S-(−)-1-(3-methoxyphenyl)ethanamine L (+) MA salt to S-(−)-1-(3-methoxyphenyl)ethanamine.

In another embodiment, the present invention provides a process for separation of S(−)-N,N-dimethyl-3-hydroxy-3-(2-thienyl)propanamine from a first mixture of isomers, which process includes:

a) reacting the first mixture of isomers with D-(−) MA to recover the diastereomeric salt of R-(+)-N,N-dimethyl-3-hydroxy-3-(2-thienyl propanamine D-(−) MA as solid;

b) reacting the second mixture of isomers obtained from the mother liquors of step a) with L (+) MA to form S-(−)-N,N-dimethyl-3-hydroxy-3-(2-thienyl)propanamine L (+) MA as solid; and

c) converting the S-(−)-N,N-dimethyl-3-hydroxy-3-(2-thienyl)propanamine mandalate salt to S-(−)-N,N-dimethyl-3-hydroxy-3-(2-thienyl)propanamine.

In yet another embodiment, the present application provide a process for separation of (−)4-(4-(dimethylamino)-1-(4-fluorophenyl)-1-hydroxybutyl)-3-(hydroxymethyl)benzonitrile (escitalopram diol) from a first mixture of isomers, which process includes

a) reacting the first mixture of isomers with (−) DPTTA to obtain (+)4-(4-(dimethylamino)-1-(4-fluorophenyl)-1-hydroxybutyl)-3-(hydroxymethyl)benzonitrile (−) DPTTA salt as solid;

b) reacting the second mixture of isomers obtained from the mother liquors of step a) with (+) DPTTA to form escitalopram diol (+) DPTTA salt as solid; and

c) converting the escitalopram diol (+) DPTTA salt to Escitalopram of Formula III or a salt thereof.

The process of the present application may be utilized for separation of enantiomerically pure compounds from their mixtures of isomers for most of the chiral active pharmaceutical ingredients including but not limited to Clopidogrel, Repaglinide, R-modafinil, Cinalcalcet, Escitalopram, Sitagliptin, Voriconazole and optically pure intermediates used for their preparation.

It is with in the scope of the present application that one skilled in the art may make certain modifications depending on the nature and requirements to the process of the present application while applying it to the other compounds.

In another aspect the present invention relates to the method of using the enantiomerically pure intermediates obtained according to the process of present application in the preparation of active pharmaceutical ingredients including Rivastigmine, Duloxetine and Escitalopram or a salt thereof.

The certain embodiments of the present invention are illustrated in the following examples

### EXAMPLES

### Reference Example 1

### Preparation of (−)-3-[(1-dimethylamino)ethyl]-phenyl-N-ethyl-N-methyl-carbamate (+)-DPTTA salt

Step (a): 74 gm of (±)-3-[(1-dimethylamino)ethyl]-phenyl-N-ethyl-N-methyl-carbamate was dissolved in methanol (370 ml) and water (185 ml) and stirred. 96 gm of (+)-DPTTA was added to the solution. The solution starts to crystallize at 5-10° C. The solid was filtered to give 148 gm of required salt in the form of white crystals. The salt was further purified with the mixture of water (75 ml) and methanol (150 ml) to obtain 80 gm of wet solid. The purification process was repeated for 4 more times to obtain 42 gm of the required salt.

Step (b): The mother liquor obtained in the resolution as well as purifications in step a) was distilled completely. The residue was treated with aqueous sodium hydroxide in a mixture of water and toluene. The organic layer was separated and distilled completely to obtain 75 gm of residue.

The residue was dissolved in methanol (375 ml) and water (187.5 ml) and stirred. (+)-DPTTA (75 gm) was charged to the solution. The precipitated solid was filtered to obtain 36.5 gm of the desired salt. The salt was further purified with mixture of water (18 ml) and methanol (36 ml) to give 20 gm of the desired salt. The purification process was repeated for 4 more times to obtain 9 gm of the required salt. The total yield of the title compound is 51 gm.

### Example 1

### Preparation of (−)-3-[(1-dimethylamino)ethyl]-phenyl-N-ethyl-N-methyl-carbamate (+) DPTTA salt

Step (a): 74 gm of (±)-3-[(1-dimethylamino)ethyl]-phenyl-N-ethyl-N-methyl-carbamate was dissolved in methanol (370 ml) and water (185 ml) and stirred. 96 gm of (+)-DPTTA was added to the solution. The solution starts to crystallize at 5-10° C. The solid was filtered to give 148 gm of required salt in the form of white crystals. The salt was further purified with the mixture of water (75 ml) and methanol (150 ml) to obtain 80 gm of wet solid. The purification process was repeated for 4 more times to obtain 43 gm of the required salt.

Step (b): The mother liquor obtained in the resolution as well as purifications in step a) was distilled completely. The residue was treated with aqueous sodium hydroxide in a mixture of water and toluene. The organic layer was separated and distilled completely to obtain 75 gm of first mixture of isomers as residue.

The residue obtained above was dissolved in methanol (375 ml) and water (187.5 ml) and stirred. (−)-DPTTA (75 gm) was charged to the solution. The precipitated solid was filtered to obtain 80 gm of the unwanted salt. The salt was further recrystallized in mixture of water (40 ml) and methanol (80 ml) to give 70 gm of the unwanted salt. The recrystallization process was repeated for 2 more times to obtain 35 gm of the unwanted salt

Step (c): The mother liquor obtained in the resolution as well as purifications in step b) was distilled completely. The residue was treated with aqueous sodium hydroxide in a mixture of water and toluene. The organic layer was separated and distilled completely to obtain 40 gm second mixture of isomers as residue.

The residue was dissolved in methanol (200 ml) and water (100 ml) and charged with the (+)-DPTTA (40 gm). The solution starts to crystallize at 5-10° C. The solid was filtered to give 60 gm of required salt in the form of white crystals. The salt was further purified with the mixture of water (30 ml) and methanol (60 ml) to obtain 45 gm of wet solid. The purification process was repeated for 3 more times to obtain 22 gm (2^{nd} crop) of the required salt as dry material.

The overall yield of the title compound is 65 gm.

### Example 2

### Preparation Rivastigmine Hydrogentartarate Salt

22 gm of the Rivastigmine (+) DPTTA salt obtained as 2^{nd} crop in Example 1 Step (c) was charged in a flask containing 150 ml of water and pH was adjusted to about 11 using about 20 ml of aqueous ammonia. The reaction mixture was extracted with dichloromethane (DCM) (250 ml) and the organic layer was washed with water (100 ml). The final organic layer was distilled completely to obtain 9.5 gm of Rivastigmine base as residue.

25 ml of acetone was added to the above obtained residue and stirred for dissolution. 5.7 gm of L (+) tartaric acid was added to the solution, heated to about 50° C. and stirred for about 30 minutes. The reaction mixture was cooled to 10-15° C. and stirred for 1 hour. The solid was filtered and washed with acetone (10 ml). The solid was dried 50-60° C. to obtain 12.5 gm of the title compound.

### Reference Example 2

### Preparation of S-(−)-1-(3-methoxyphenyl)ethanamine(+)-mandelic acid

Step (a): 89.5 gm of (±)-1-(3-methoxyphenyl)ethanamine was dissolved in isopropyl alcohol (3135 ml) and 89.5 gm of L (+)-Mandelic acid was added to the solution. The solution was heated to reflux and stirred for 10 minutes and cooled to 33-37° C. The reaction mixture was maintained for 15 min at 33-37° C. The solid was filtered and washed with isopropyl alcohol (20 ml). The wet solid (70 gm) was charged in isopropyl alcohol (1050 ml), heated to reflux and stirred for 10 minutes. The reaction mixture was cooled to 33-37° C. and maintained for 15 min at 33-37° C. The solid was filtered and washed with isopropyl alcohol (20 ml) to obtain 51.5 gm of required isomer as salt.

Step (b): The mother liquor obtained in the resolution as well as purifications in step a) was distilled completely to obtain 61 gm of residue. 500 ml of water and 200 ml of ethyl acetate was added to the residue and pH adjusted to about 11 using caustic lye (15 ml). The aqueous layer was separated and extracted with ethyl acetate (2×100 ml). Total organic layer was washed with 10% aqueous NaCl solution (150 ml). The final organic layer was distilled completely to obtain 30 gm of residue.

The residue was dissolved in isopropyl alcohol (1150 ml) and L (+)-Mandelic acid (30 gm) was charged to the solution. The solution was heated to reflux and stirred for 10 minutes and cooled to 33-37° C. The reaction mixture was maintained for 15 min at 33-37° C. The solid was filtered and washed with isopropyl alcohol (20 ml). The wet solid (12 gm) was charged in isopropyl alcohol (180 ml), heated to reflux and stirred for 10 minutes. The reaction mixture was cooled to 33-37° C. and maintained for 15 min at 33-37° C. The solid was filtered and washed with isopropyl alcohol (10 ml) to obtain 8 gm of required isomer as salt.

Thus resulting in the overall yield of 59.5 gm

### Example 3

### Preparation of S-(−)-1-(3-methoxyphenyl)ethanamine (+)-mandelic acid

Step (a): 89.5 gm of (±)-1-(3-methoxyphenyl)ethanamine was dissolved in isopropyl alcohol (3135 ml) and 89.5 gm of L (+)-Mandelic acid was added to the solution. The solution was heated to reflux and stirred for 10 minutes and cooled to 33-37° C. The reaction mixture was maintained for 15 min at 33-37° C. The solid was filtered and washed with isopropyl alcohol (20 ml). The wet solid (70 gm) was charged in isopropyl alcohol (1050 ml), heated to reflux and stirred for 10 minutes. The reaction mixture was cooled to 33-37° C. and maintained for 15 min at 33-37° C. The solid was filtered and washed with isopropyl alcohol (20 ml) to obtain 52.5 gm of required isomer as salt.

Step (b): The mother liquor obtained in the resolution as well as purifications in step a) was distilled completely to obtain 61 gm of residue. 500 ml of water and 200 ml of ethyl acetate was added to the residue and pH adjusted to about 11 using caustic lye (15 ml). The aqueous layer was separated and extracted with ethyl acetate (2×100 ml). Total organic layer was washed with 10% aqueous NaCl solution (150 ml). The final organic layer was distilled completely to obtain 30 gm of residue.

The residue was dissolved in isopropyl alcohol (1060 ml) and D-(−)-Mandelic acid (30 gm) was charged to the solution. The solution was heated to reflux and stirred for 10 minutes and cooled to 33-37° C. The reaction mixture was maintained for 15 min at 33-37° C. The solid was filtered and washed with isopropyl alcohol (20 ml). The wet solid (116 gm) was charged in isopropyl alcohol (1650 ml), heated to reflux and stirred for 10 minutes. The reaction mixture was cooled to 33-37° C. and maintained for 15 min at 33-37° C. The solid was filtered and washed with isopropyl alcohol (20 ml) to obtain 58 gm of unwanted isomer as salt.

Step (c): The mother liquor obtained in the resolution as well as purifications in step b) was distilled completely to obtain 48 gm of residue. 400 ml of water and 200 ml of ethyl acetate was added to the residue and pH adjusted to about 11 using caustic lye (15 ml). The aqueous layer was separated and extracted with ethyl acetate (2×100 ml). Total organic layer was washed with 10% aqueous NaCl solution (150 ml). The final organic layer was distilled completely to obtain 30.5 gm of residue.

The residue was dissolved in isopropyl alcohol (1060 ml) and L (+)-Mandelic acid (30.5 gm) was charged to the solution. The solution was heated to reflux and stirred for 10 minutes and cooled to 33-37° C. The reaction mixture was maintained for 15 min at 33-37° C. The solid was filtered and washed with isopropyl alcohol (20 ml). The wet solid (25 gm) was charged in isopropyl alcohol (375 ml), heated to reflux and stirred for 10 minutes. The reaction mixture was cooled to 33-37° C. and maintained for 15 min at 33-37° C. The solid was filtered and washed with isopropyl alcohol (10 ml) to obtain 14 gm of required isomer as salt.

Thus resulting in the overall yield of 66.5 gm

### Example 4

### Preparation of S(−)-N,N-dimethyl-3-hydroxy-3-(2-thienyl)propanamine L (+)-MA salt

Step (a): 50 gm of (±)-N,N-dimethyl-3-hydroxy-3-(2-thienyl)propanamine was dissolved in ethyl acetate (500 ml) and heated to about 50° C. 25 gm of L (+)-MA was added to the solution and stirred for about 30 minutes. The solution starts to crystallize at about 50° C. The reaction mixture was cooled to 25-35° C. and stirred for about 1 hour. The solid was filtered and washed with ethyl acetate (20 ml). The wet solid was dried to give 40 gm of required salt in the form of white crystals. The salt was further purified by recrystallizing from ethyl acetate (150 ml) to obtain 37 gm of dry solid.

Step (b): The mother liquor obtained in the resolution as well as purification in step a) was distilled completely. The residue was treated with aqueous sodium hydroxide in a mixture of water and toluene. The organic layer was separated and distilled completely to obtain 26 gm of first mixture of isomers as residue.

The residue obtained above was dissolved in ethyl acetate (260 ml) and heated to about 50° C. 20 gm of D-(−)-MA was added to the solution and stirred for about 30 minutes. The solution starts to crystallize at about 50° C. The reaction mixture was cooled to 25-35° C. and stirred for about 1 hour. The solid was filtered and washed with ethyl acetate (10 ml) to get 60 gm of the wet solid. The wet solid was further purified two times by recrystallizing from ethyl acetate (180 ml) to obtain 37.5 gm of dry solid (unwanted).

Step (c): The mother liquor obtained in the resolution as well as purification in step b) was distilled completely. The residue was treated with aqueous sodium hydroxide in a mixture of water and toluene. The organic layer was separated and distilled completely to obtain 10 gm of second mixture of isomers as residue.

The residue obtained above was dissolved in ethyl acetate (100 ml) and heated to about 50° C. 8 gm of L (+)-MA was added to the solution and stirred for about 30 minutes. The solution starts to crystallize at about 50° C. The reaction mixture was cooled to 25-35° C. and stirred for about 1 hour. The solid was filtered and washed with ethyl acetate (10 ml) to get 10 gm of the wet solid. The wet solid was further purified two times by recrystallizing from ethyl acetate (30 ml) to obtain 6 gm of dry solid.

The overall yield of the title compound is 43 gm.

### Example 5

### Preparation of (−)4-(4-(dimethylamino)-1-(4-fluorophenyl)-1-hydroxybutyl)-3-(hydroxymethyl)benzonitrile (+) DPTTA salt

Step (a): 500 ml of isopropyl alcohol was charged to 100 gm of (+)4-(4-(dimethylamino)-1-(4-fluorophenyl)-1-hydroxybutyl)-3-(hydroxymethyl)benzonitrile (first mixture of isomers) and stirred to dissolve. 50 gm of (−)-DPTTA was added to the above solution and heated to about 40° C. The reaction mixture was stirred for one hour and then cooled to room temperature. The reaction suspension was stirred for about 2 hours for complete isolation of the solid. The reaction mixture was filtered and washed with IPA (20 ml). The wet solid was charged in 200 ml of IPA, heated to reflux and stirred for about 30 minutes. The reaction mixture was cooled to room temperature and stirred for about 30 minutes, filtered the solid and washed with IPA (20 ml). The wet solid was dried to obtain 54 gm of the unwanted salt.

Step (b): The mother liquor obtained in the resolution as well as purification in step a) was distilled completely. The residue was treated with aqueous ammonia in a mixture of water and toluene. The organic layer was separated and distilled completely to obtain 60 gm of second mixture of isomers as residue.

300 ml of isopropyl alcohol was charged to the above residue and stirred to dissolve. 30 gm of (+)-DPTTA was added to the above solution and heated to about 40° C. The reaction mixture was stirred for one hour and then cooled to room temperature. The reaction suspension was stirred for about 2 hours for complete isolation of the solid. The reaction mixture was filtered and washed with IPA (20 ml). The wet solid was charged in 200 ml of IPA, heated to reflux and stirred for about 30 minutes. The reaction mixture was cooled to room temperature and stirred for about 30 minutes, filtered the solid and washed with IPA (20 ml). The wet solid was dried to obtain 64 gm of title compound as solid.

1. A process for separation of a required isomer from a first mixture of isomers, that includes:
a) reacting the first mixture of isomers with a first optically pure acid or base to recover a first diastereomeric salt of unwanted isomer as solid;
b) reacting a second mixture of isomers obtained from the mother liquors in step a) with a second optically pure acid or base having opposite rotation with respect to the first optically pure add or base to form a second diastereomeric salt of required isomer as solid; and
c) converting the second diastereomeric salt to the required isomer.

2. The process of claim 1, wherein the first mixture of isomers comprises of about 50% or more of the unwanted isomer.

3. The process of claim 1, wherein the first mixture of isomers comprises about 60% or more of the unwanted isomer.

4. The process of claim 1, wherein the second mixture of isomers comprises about 60% or more of the required isomer.

5. The process of claim 1, wherein the first mixture of isomers is obtained by an asymmetric synthesis or a stereo selective synthesis.

6. The process of claim 1, wherein the first mixture of isomers is obtained by processing the mother liquors obtained during the resolution of a racemic mixture using an optically pure acid or base.

7. The process of claim 1, wherein said optically pure acid comprises mandelic acid, tartaric acid, di-p-toluyl tartaric acid, dibenzoyl tartaric acid, and camphor sulfonic acid.

8. The process of claim 1, wherein said optically pure base comprises 1-phenylethylamine, ephedrine, 2-amino-1-butanol, 2-amino-1-phenyl-1,3-propanediol, 1-naphthyl-1-ethylamine, (−)-quinine, (+)-quinidine, (−)-brucine and (+)-dehydroabietylamine N-octyl-D-glucomine.

9. The process of claim 1, wherein the reaction is carried out in a solvent comprises of methanol, ethanol or isopropanol; acetone, methyl ethyl ketone or methyl isoburyl ketone.

10. The process of claim 1, wherein the reaction temperature range is from about 20° C. to 80° C.

11. The process of claim 1, wherein the reaction of step c) comprises treating the second diasteromeric salt with a base or an acid.

12. The process of claim 11, wherein the base comprises lithium hydroxide, sodium hydroxide, potassium hydroxide, sodium carbonate, potassium carbonate, sodium bicarbonate, potassium bicarbonate, ammonia; and mixtures thereof.

13. The process of claim 11, wherein the acid comprises hydrochloric acid, sulfuric acid, and acetic acid.

14. A process for separation of Rivastigmine from a first mixture of isomers which process includes:
a) reacting the first mixture of isomers with (−) DPTTA to recover (R)-Rivastigmine (−) DPTTA salt as solid;
b) reacting a second mixture of isomers obtained from the mother liquors in step a) with (+) DPTTA to form Rivastigmine (+) DPTTA salt as solid; and
c) converting the Rivastigmine (+) DPTTA salt to Rivastigmine.

15. The process of claim 14, wherein the reaction is carried out in a solvent selected from methanol, ethanol, isopropanol, acetone, methyl ethyl ketone, methyl isobutyl ketone, and ethyl acetate.

16. The process of claim 14, wherein the reaction of step c) comprises treating the Rivastigmine (+) DPTTA salt with a base.

17. The process of claim 16, wherein said base is selected from lithium hydroxide, sodium hydroxide, potassium hydroxide, sodium carbonate, potassium carbonate, sodium bicarbonate, potassium bicarbonate, ammonia; and mixtures thereof.

18. The process of claim 14 which further comprises converting Rivastigmine to a pharmaceutically acceptable salt.

19. A process for separation of S-(−)-1-(3-methoxyphenyl)ethanamine from a first mixture of isomers, which process includes:
a) reacting the first mixture of isomers with D-(−) Mandelic acid (MA) to recover R-(+)-1-(3-methoxyphenyl)ethanamine D-(−) MA salt as solid;
b) reacting a second mixture of isomers obtained from the mother liquors in step a) with L (+) MA to form S-(−)-1-(3-methoxyphenyl)ethanamine L (+) MA as solid; and
c) converting the S-(−)-1-(3-methoxyphenyl)ethanamine L (+) MA salt to S-(−)-1-(3-methoxyphenyl)ethanamine.

20. A process for separation of S-(−)-N,N-dimethyl-3-hydroxy-3-(2-thienyl)propanamine from a first mixture of isomers, which process includes:
a) reacting the first mixture of isomers with D-(−) MA to recover R-(+)-N,N-dimethyl-3-hydroxy-3-(2-thienyl propanamine D-(−) MA salt as solid;
b) reacting a second mixture of isomers obtained from the mother liquors of step a) with L (+) MA to form S-(−)-N,N-dimethyl-3-hydroxy-3-(2-thienyl)propanamine L (+) MA salt as solid; and
c) converting the S-(−)-N,N-dimethyl-3-hydroxy-3-(2-thienyl)propanamine L (+) MA salt to S-(−)-N,N-dimethyl-3-hydroxy-3-(2-thienyl)propanamine.

21. A process for separation of (−)4-(4-(dimethylamino)-1-(4-fluorophenyl)-1-hydroxybutyl)-3-(hydroxymethyl)benzonitrile (escitalopram diol) from a first mixture of isomers, which process includes
a) reacting the first mixture of isomers with (−) DPTTA to obtain (+)4-(4-(dimethylamino)-1-(4-fluorophenyl)-1-hydroxybutyl)-3-(hydroxymethyl)benzonitrile (−) DPTTA salt as solid;
b) reacting a second mixture of isomers obtained from the mother liquors of step a) with (+) DPTTA to form escitalopram diol (+) DPTTA salt as solid; and
c) converting the escitalopram diol (+) DPTTA salt to Escitalopram or a salt thereof.

22. (canceled)

23. A method of using the enantiomerically pure intermediates obtained according to the process of present application in the preparation of active pharmaceutical ingredients including Rivastigmine, Duloxetine, Escitalopram or a salt thereof.

24. The process of claim 18 wherein said salt is hydrogentartrate salt.
